(19) United States
(12) Patent Application Publication
(10) Pub. No.: US 2019/0077966 A1
(43) Pub. Date: Mar. 14, 2019

(54) PROTEIN ADHESION INHIBITOR, CURED PRODUCT, METHOD FOR PRODUCING CURED PRODUCT, AND ARTICLE

(71) Applicant: AGC Inc., Chiyoda-ku (JP)

(72) Inventors: Ryohei KOGUCHI, Chiyoda-ku (JP); Kyoko Yamamoto, Chiyoda-ku (JP)

(73) Assignee: AGC Inc., Chiyoda-ku (JP)

(21) Appl. No.: 16/188,358

(22) Filed: Nov. 13, 2018

Related U.S. Application Data
(63) Continuation of application No. PCT/JP2017/019594, filed on May 25, 2017.

(30) Foreign Application Priority Data
May 27, 2016 (JP) 2016-106136

(51) Int. Cl. C09D 4/06; C09D 127/12; A61L 31/10; A61L 31/04; A61L 29/08; A61L 29/04; A61L 27/34; A61L 27/26; A61L 27/16

(52) U.S. Cl. CPC C09D 4/06; C09D 127/12; A61L 31/10; A61L 31/041; A61L 29/085; A61L 2420/06; A61L 29/041; A61L 27/34; A61L 27/26; A61L 27/16; A61L 2420/02; A61L 29/049

(57) ABSTRACT

To provide a protein adhesion inhibitor from which a cured product having excellent protein non-adhesion and having excellent form stability so that a film formed therefrom will not warp, can be formed, a cured product using it, and an article.

A protein adhesion inhibitor comprising a non-polymerizable fluorinated polymer having a specific group such as —(CₙH₂ₙO)— and a fluorine atom content Q_{F} of from 5 to 60 mass %, and at least one curable monomer selected from the group consisting of a vinyl monomer and a cyclic ether monomer, wherein the coefficient α is at most 10. A cured product of the protein adhesion inhibitor. A medical device 1 comprising a substrate 2 and a coating layer 3 formed of the cured product of the protein adhesion inhibitor on the substrate 2.

### TECHNICAL FIELD

The present invention relates to a protein adhesion inhibitor, a cured product, a method for producing a cured product, and an article.

### BACKGROUND ART

In recent years, regenerative medicine is developing for the purpose of regenerating functions actively utilizing cells. In the field of regenerative medicine, culture and proliferation of cells are carried out using a cell culture vessel in vitro. However, by conventional culture in vitro, biological components such as proteins and blood cells are likely to be adsorbed on the surface of the vessel, and accordingly it is difficult to conduct culture and proliferation in the same manner as the cell proliferation in vivo, e.g. by three-dimensional culture.

As a method for suppressing adsorption of proteins, a method has been proposed to use a protein adhesion inhibitor which contains a fluorinated polymer having a structure similar to a biological membrane such as a polyethylene glycol chain and having a fluorine atom content of from 5 to 60 mass %, and a non-fluorinated curable monomer (Patent Document 1).

### PRIOR ART DOCUMENTS

### Patent Documents

Patent Document 1: WO2016/010147

### DISCLOSURE OF INVENTION

### Technical Problem

Patent Document 1 discloses that a film is formed by using the above specific protein adhesion inhibitor and the film is bonded to a device surface, whereby adsorption of proteins can be suppressed. However, with the protein adhesion inhibitor in Patent Document 1, when a film is formed by curing particularly by UV irradiation, the film may be warped and can hardly be bonded to the device surface, and its problem is not small practically.

The object of the present invention is to provide a protein adhesion inhibitor from which a cured product having excellent protein non-adsorption and having excellent form stability so that a film formed therefrom will not warp, can be formed, a cured product using the protein adhesion inhibitor and its production method, and an article.

### Solution to Problem

The present invention provides a protein adhesion inhibitor, a cured product, a method for producing a cured product, and an article, having the following constitution.

[1] A protein adhesion inhibitor comprising a non-polymerizable fluorinated polymer having at least one group selected from the group consisting of a group represented by the following formula (1), a group represented by the following formula (2) and a group represented by the following formula (3) and having a fluorine atom content Q_{F} of from 5 to 60 mass %, and

at least one curable monomer selected from the group consisting of a vinyl monomer and a cyclic ether monomer,

wherein the coefficient α represented by the following formula (I) is at most 10:

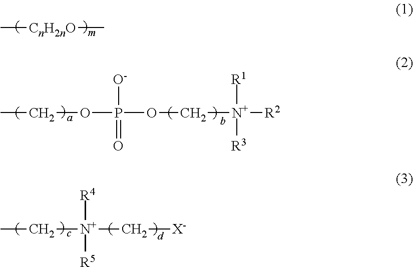

wherein n is an integer of from 1 to 10, m is an integer of from 1 to 100 in a case where the group represented by the formula (1) is contained in a side chain of the non-polymerizable fluorinated polymer or from 5 to 300 in a case where contained in the main chain, R¹ to R³ are each independently a C₁₋₅ alkyl group, “a” is an integer of from 1 to 5, b is an integer of from 1 to 5, R⁴ and R⁵ are each independently a C₁₋₅ alkyl group, X⁻ is a group represented by the following formula (3-1) or a group represented by the following formula (3-2), c is an integer of from 1 to 20, and d is an integer of from 1 to 5;

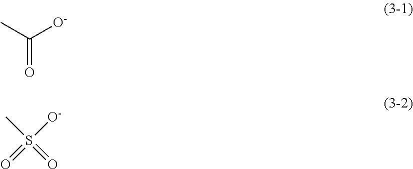

$$α = ( \frac{1000}{{M}_{1}} \times {N}_{1} \times \frac{{W}_{1}}{100} ) \times 2 + ( \frac{1000}{{M}_{2}} \times {N}_{2} \times \frac{{W}_{2}}{100} ) \qquad ( I )$$

wherein M₁ is the molecular weight of the vinyl monomer, N₁ is the number of polymerizable functional groups in the vinyl monomer, W₁ is the content (mass %) of the vinyl monomer to the total mass of the protein adhesion inhibitor, M₂ is the molecular weight of the cyclic ether monomer, N₂ is the number of polymerizable functional groups in the cyclic ether monomer, and W₂ is the content (mass %) of the cyclic ether monomer to the total mass of the protein adhesion inhibitor.
[2] The protein adhesion inhibitor according to [1], wherein the content of the curable monomer is from 50.00 to 99.99 mass %.
[3] The protein adhesion inhibitor according to [1] or [2], wherein the vinyl monomer is contained as the curable monomer, and the number of polymerizable functional groups in the vinyl monomer is from 1 to 20.
[4] The protein adhesion inhibitor according to any one of [1] to [3], wherein the vinyl monomer is contained as the curable monomer, and the molecular weight of the vinyl monomer is from 100 to 100,000.
[5] The protein adhesion inhibitor according to any one of [1] to [4], wherein the cyclic ether monomer is contained as the curable monomer, and the number of polymerizable functional groups in the cyclic ether monomer is from 1 to 20.
[6] The protein adhesion inhibitor according to any one of [1] to [5], wherein the cyclic ether monomer is contained as the curable monomer, and the molecular weight of the cyclic ether monomer is from 50 to 50,000.
[7] A cured product of the protein adhesion inhibitor as defined in any one of [1] to [6].
[8] The cured product according to [7], which has a convexo-concave pattern formed on its surface.
[9] A method for producing a cured product, which comprises irradiating a coating film formed from a coating solution containing the protein adhesion inhibitor as defined in any one of [1] to [6] with light in such a state that a convexo-concave plane formed on the surface of a mold is pressed against the coating film, thereby to cure the coating film to obtain a film-form cured product.
[10] An article having the cured product as defined in [7] or [8] on at least a part of its surface.
[11] The article according to [10], which comprises a substrate and a coating layer formed of the cured product on the substrate.
[12] The article according to [10] or [11], which is a medical device.

### Advantageous Effects of Invention

According to the protein adhesion inhibitor of the present invention, it is possible to form a cured product having excellent protein non-adsorption and having excellent form stability so that a film formed therefrom will not warp. The cured product of the present invention has excellent protein non-adsorption and form stability.

According to the method for producing a cured product of the present invention, a cured product having excellent protein non-adsorption and form stability can be produced. The article of the present invention has excellent protein non-adsorption and form stability.

### BRIEF DESCRIPTION OF DRAWINGS

FIG. 1 is a view schematically illustrating steps in an example of the method for producing a cured product of the present invention.

FIG. 2 is a view schematically illustrating an example of a medical device as the article of the present invention.

FIG. 3 is an I-I cross section of the medical device in FIG. 2

FIG. 4 is a perspective view illustrating another example of a medical device as the article of the present invention.

### DESCRIPTION OF EMBODIMENTS

The following definitions of terms and method of use apply throughout the present specification including claims.

A “non-polymerizable fluorinated polymer” means a polymer compound having fluorine atom(s) in its molecule and having no polymerizable functional group.

A “polymerizable functional group” means a functional group which contributes to addition polymerization or ring-opening polymerization. The polymerizable functional group may, for example, be a vinyl group, a (meth)acryloyl group, an epoxy group or an oxetane group.

A “glass transition temperature (Tg)” of a polymer means an intermediate glass transition temperature for a change from the rubbery state to the glass state, as measured by a differential scanning calorimetry (DSC) method.

A “number average molecular weight (Mn)” and a “mass average molecular weight (Mw)” of a polymer mean values as calculated as polystyrene by gel permeation chromatography (GPC).

A “unit” means a polymer unit derived from a monomer, which is present in a polymer and which constitutes the polymer. A unit derived from a monomer having a carbon-carbon unsaturated double bond, formed by addition polymerization of the monomer, is a divalent unit formed by cleavage of the unsaturated double bond. Further, one obtained by chemically converting the structure of a certain unit after formation of a polymer will also be referred to as a unit.

A “curable monomer” means a compound having at least one polymerizable functional group in its molecule.

A “vinyl monomer” means a compound having at least one polymerizable unsaturated group such as a vinyl group or a (meth)acryloyl group in its molecule and having no ring-opening polymerizable cyclic ether group.

A “cyclic ether monomer” means a compound having at least one ring-opening polymerizable cyclic ether group such as an epoxy group or an oxetane group in its molecule and having no polymerizable unsaturated group.

A “(meth)acryloyl group” is a generic term for an acryloyl group and a methacryloyl group, and a “(meth)acrylate” is a generic term for an acrylate and a methacrylate.

A “medical device” is a device used for a medical purpose such as therapeutic, diagnostic, anatomical or biological examination, and includes a device to be inserted or in contact with a living body such as a human body, or to be in contact with a medium (such as blood) taken out from a living body.

A “group represented by the formula (1)” will sometimes be referred to as “a group (1)”. The same applies to groups represented by other formulae.

### [Protein Adhesion Inhibitor]

The protein adhesion inhibitor of the present invention is a composition to impart protein non-adsorption to the surface of an article. The protein adhesion inhibitor of the present invention comprises a non-polymerizable fluorinated polymer (hereinafter referred to as a “fluorinated polymer (A)”) having at least one group selected from the group consisting of a group (1), a group (2) and a group (3) and having a fluorine atom content Q_{F} of from 5 to 60 mass %, and at least one curable monomer (hereinafter referred to as a “curable monomer (B)”) selected from the group consisting of a vinyl monomer and a cyclic ether monomer:

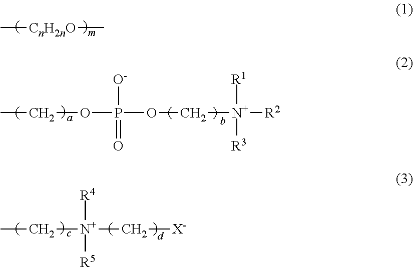

wherein the symbols are as defined above.

### (Fluorinated Polymer (A))

### Group (1):

The group (1) may be contained in the main chain of the fluorinated polymer (A), or may be contained in a side chain. The group (1) may be linear or branched. From the viewpoint of a higher protein adsorption inhibitory effect, the group (1) is preferably linear.

n is preferably an integer of from 1 to 6, more preferably an integer of from 1 to 4, from such a viewpoint that protein is thereby less likely to be adsorbed.

In a case where the group (1) is contained in a side chain of the fluorinated polymer (A), m is preferably from 1 to 40, particularly from 1 to 20, from the viewpoint of excellent water resistance. In a case where the group (1) is contained in the main chain of the fluorinated polymer (A), m is preferably from 10 to 200, from the viewpoint of excellent water resistance.

In a case where m is 2 or more, the plurality of (CₙH₂ₙO) in the group (1) may be of one type, or may be of two or more types. Further, in the case of two or more types, their disposition may be either random, block or alternating. In a case where n is 3 or more, (CₙH₂ₙO) may be a straight-chain structure or a branched structure.

In a case where the fluorinated polymer (A) has groups (1), the groups (1) in the fluorinated polymer (A) may be of one type, or of two or more types.

### Group (2):

The group (2) is preferably contained in a side chain of the fluorinated polymer (A).

R¹ to R³ are each independently a C₁₋₅ alkyl group, and from the viewpoint of easy availability of raw material, preferably a C₁₋₄ alkyl group, particularly preferably a methyl group.

“a” is an integer of from 1 to 5, and from the viewpoint of easy availability of raw material, preferably an integer of from 2 to 5, particularly preferably 2.

b is an integer of from 1 to 5, and from such a viewpoint that protein is less likely to be adsorbed, preferably an integer of from 1 to 4, particularly preferably 2.

In a case where the fluorinated polymer (A) has groups (2), the groups (2) in the fluorinated polymer (A) may be of one type, or of two or more types.

### Group (3):

The group (3) is preferably contained in a side chain of the fluorinated polymer (A).

R⁴ and R⁵ are each independently a C₁₋₅ alkyl group, and from such a viewpoint that protein is less likely to be adsorbed, preferably a C₁₋₄ alkyl group, particularly preferably a methyl group.

c is an integer of from 1 to 20, and from such a viewpoint that the fluorinated polymer (A) will be excellent in flexibility, preferably an integer of from 1 to 15, more preferably an integer of from 1 to 10, particularly preferably 2.

d is an integer of from 1 to 5, and from such a viewpoint that protein is less likely to be adsorbed, preferably an integer of from 1 to 4, particularly preferably 1.

In a case where the fluorinated polymer (A) has groups (3), the groups (3) may be of one type, or of two or more types. Further, from such a viewpoint that protein is less likely to be adsorbed, it is preferred that the fluorinated polymer (A) has either groups (3) wherein X⁻ is a group (3-1), or groups (3) wherein X⁻ is a group (3-2).

The fluorinated polymer (A) preferably has units having any one of groups (1) to (3) and having no fluorine atom and units having a fluorine atom(s) and having no group (1) to (3), from such a viewpoint that protein is still less likely to be adsorbed on the surface of the article.

The proportion of the units having a fluorine atom(s) and having no group (1) to (3) is preferably more than 10 mol % to all the units of the fluorinated polymer (A). When the proportion of such units is more than 10 mol %, the surface tension of the surface of the resulting article will be sufficiently low. The proportion of such units is more preferably more than 10 mol % and at most 95 mol %, particularly preferably more than 10 mol % and at most 90 mol %. When the proportion of such units is at most the upper limit value of the above range, protein is less likely to be adsorbed on the surface of the article.

The proportion of the units having any one of the groups (1) to (3) and having no fluorine atom is preferably less than 90 mol % to all the units of the fluorinated polymer (A). When the proportion of such units is less than 90 mol %, water resistance of the surface of the article will be excellent. The proportion of such units is more preferably at least 5 mol % and less than 90 mol %, particularly preferably at least 10 mol % and less than 90 mol %. When the proportion of such units is at least the lower limit value of the above range, protein is less likely to be adsorbed on the surface of the article.

The fluorine atom content Q_{F} of the fluorinated polymer (A) is from 5 to 60 mass %, preferably from 5 to 55 mass %, particularly preferably from 5 to 50 mass %. The higher the fluorine atom content Q_{F}, the higher the surface migration property of the fluorinated polymer (A), and accordingly protein non-adsorption will efficiently develop on the surface of a cured product even when the content of the fluorinated polymer (A) is low. When the fluorine atom content Q_{F} is at least the lower limit value of the above range, the fluorinated polymer (A) tends to be unevenly present in the vicinity of the surface of a cured product, whereby excellent protein non-adsorption is likely to be obtained, and the water resistance on the surface of the article will be excellent. When the fluorine atom content Q_{F} is at most the upper limit value of the above range, protein is less likely to be adsorbed on the surface of the article.

Here, the fluorine atom content Q_{F} (mass %) is determined by the following formula.

Q_{F}=[19×N_{F}/M_{A}]×100

N_{F}: the sum of values obtained by multiplying, for every type of units that constitute the fluorinated polymer (A), the number of fluorine atoms in the unit by the molar ratio of the unit to all units.

M_{A}: the sum of values obtained by multiplying, for every type of units that constitute the fluorinated polymer (A), the total atomic weight of all atoms constituting the unit by the molar ratio of the unit to all units.

For example, in the case of a fluorinated polymer having 50 mol % of tetrafluoroethylene (TFE) units and 50 mol % of ethylene (E) units, Q_{F} is as follows.

The value obtained by multiplying the number of fluorine atoms (4) in a TFE unit by the molar ratio (0.5) of the TFE unit to all units, is 2, and the value obtained by multiplying the number of fluorine atoms (0) in an E unit by the molar ratio (0.5) of the E unit, is 0, and therefore, N_{F} is 2. The value obtained by multiplying the total atomic weight (100) of all atoms constituting the TFE unit, by the molar ratio (0.5) of the TFE unit to all units, is 50, and the value obtained by multiplying the total atomic weight (28) of all atoms constituting the E unit, by the molar ratio (0.5) of the E unit to all units, is 14, and therefore, M_{A} is 64. Accordingly, the fluorine atom content Q_{F} of the fluorinated polymer becomes to be 59.4 mass %.

Further, the fluorine atom content Q_{F} can be measured by the method described in Examples. It can also be calculated from the charged amounts of the monomers and initiator used in the production of the fluorinated polymer (A).

The glass transition temperature of the fluorinated polymer (A) is preferably from −100 to 120° C., more preferably from −100 to 80° C., further preferably from −100 to 40° C., particularly preferably from −50 to 0° C. When the glass transition temperature is at least the lower limit value of the above range, the fluorinated polymer (A) has an appropriate viscosity such that it is easily formed at room temperature. When the glass transition temperature is at most the upper limit value of the above range, adsorption of proteins on the surface of the article is likely to be inhibited. Further, when the glass transition temperature is at most 40° C., flowability of the fluorinated polymer (A) at room temperature is sufficiently high, and the surface migration property is high, and accordingly excellent protein non-adsorption is likely to be obtained even at room temperature without a pretreatment to make the fluorinated polymer (A) be preliminarily brought into contact with hot water.

In order to lower the glass transition temperature of the fluorinated polymer (A), it is preferred to employ the groups (1). Since the groups (2) and (3) have both positive charge and negative charge, if the amount of such groups is large, the glass transition temperature tends to be high due to an influence by ionic bond. Since the group (1) has no positive charge nor negative charge, the glass transition temperature will not increase by ionic bond.

The number average molecular weight (Mn) of the fluorinated polymer (A) is preferably from 2,000 to 1,000,000, particularly preferably from 2,000 to 800,000. When the number average molecular weight is at least the lower limit value of the above range, durability of the resulting article will be excellent. When the number average molecular weight is at most the upper limit value of the above range, formability will be excellent.

The mass average molecular weight (Mw) of the fluorinated polymer (A) is preferably from 2,000 to 2,000,000, particularly preferably from 2,000 to 1,000,000. When the mass average molecular weight is at least the lower limit value of the above range, durability of the resulting article will be excellent. When the mass average molecular weight is at most the upper limit value of the above range, formability will be excellent.

The molecular weight distribution (Mw/Mn) of the fluorinated polymer (A) is preferably from 1 to 10, particularly preferably from 1.1 to 5. When the molecular weight distribution is within the above range, water resistance of the surface of the resulting article will be excellent, and protein is less likely to be adsorbed on the surface of the article.

As the fluorinated polymer (A), fluoropolymers (A1) and (A2) as described below are preferred from such a viewpoint that water resistance of the surface of the article is excellent and components are less likely to be eluted, and protein is less likely to be adsorbed on the surface of the article.

### <Fluorinated Polymer (A1)>

The fluorinated polymer (A1) has units (hereinafter referred to also as units (m1)) derived from the following monomer (m1) and at least one type of units selected from the group consisting of units (hereinafter referred to also as units (m2)) derived from the monomer (m2) and units (hereinafter referred to also as units (m3)) derived from the monomer (m3):

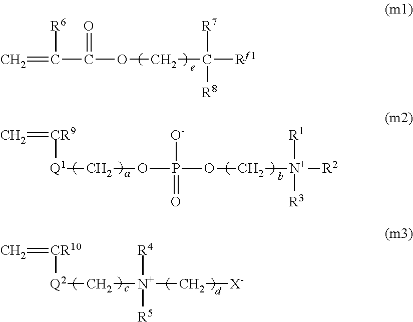

In the formula (m1), R⁶ is a hydrogen atom, a chlorine atom or a methyl group, e is an integer of from 0 to 3, R⁷ and R⁸ are each independently a hydrogen atom, a fluorine atom or a trifluoromethyl a group, and R^{f1} is a C₁₋₂₀ perfluoroalkyl group. In the formula (m2), R⁹ is a hydrogen atom, a chlorine atom or a methyl group, Q¹ is —C(═O)—O— or —C(═O)—NH—, R¹ to R³ are each independently a C₁₋₅ alkyl group, “a” is an integer of from 1 to 5, and b is an integer of from 1 to 5. In the formula (m3), R¹⁰ is a hydrogen atom, a chlorine atom or a methyl group, Q² is —C(═O)—O— or —C(═O)—NH—, R⁴ and R⁵ are each independently a C₁₋₅ alkyl group, X⁻ is the group (3-1) or the group (3-2), c is an integer of from 1 to 20, and d is an integer of from 1 to 5.

### Monomer (m1):

In the formula (m1), R⁶ is preferably a hydrogen atom or a methyl group from the viewpoint of polymerization efficiency.

e is, from the viewpoint of excellent flexibility of the fluorinated polymer (A1), preferably an integer of from 1 to 3, particularly preferably 1 or 2.

R⁷ and R⁸ are, from the viewpoint of excellent water resistance of the surface of the article, each preferably a fluorine atom.

The perfluoroalkyl group for R^{f1} may be linear or branched. As R^{f1}, from the viewpoint of easy availability of raw material, a C₁₋₁₀ perfluoroalkyl group is preferred, and a C₁₋₅ perfluoroalkyl group is particularly preferred.

As the monomer (m1), from the viewpoint of excellent water resistance of the surface of the article, CH₂═C(CH₃)COO(CH₂)₂(CF₂)₅CF₃, CH₂═CHCOO(CH₂)₂(CF₂)₅CF₃ or CH₂═CCH₃COOCH₂CF₃ is particularly preferred. Units (m1) may be of one type, or of two or more types.

### Monomer (m2):

In the formula (m2), R⁹ is preferably a hydrogen atom or a methyl group from the viewpoint of polymerization efficiency.

Q¹ is —C(═O)—O— or —C(═O)—NH—, and from such a viewpoint that protein is less likely to be adsorbed on the surface of the article, preferably —C(═O)—O—.

Specific examples of the monomer (m2) may, for example, be 2-methacryloyloxyethyl phosphorylcholine, 2-acryloyloxyethyl phosphorylcholine, etc. In a case where the fluorinated polymer (A1) has units (m2), the units (m2) may be of one type, or of two or more types.

### Monomer (m3):

In the formula (m3), R¹⁰ is preferably a hydrogen atom or a methyl group from the viewpoint of polymerization efficiency.

Q² is —C(═O)—O— or —C(═O)—NH—, and from such a viewpoint that protein is less likely to be adsorbed on the surface of the article, preferably —C(═O)—O—.

As the monomer (m3), from such a viewpoint that protein is less likely to be adsorbed on the surface of the article, N-methacryloyloxyethyl-N,N-dimethyl ammonium-α-N-methyl carboxy betaine or N-acryloyloxyethyl-N,N-dimethyl ammonium-α-N-methyl carboxy betaine is preferred. In a case where the fluorinated polymer (A1) has units (m3), the units (m3) may be of one type, or of two or more types.

From such a viewpoint that protein is less likely to be adsorbed on the surface of the article, it is particularly preferred that the fluorinated polymer (A1) has either one type of units (m2) and units (m3). Here, the fluorinated polymer (A1) may have all of units (m1), units (m2) and units (m3).

The fluorinated polymer (A1) is obtainable by carrying out a polymerization reaction of the monomers in a polymerization solvent by using a known method. The polymerization solvent may, for example, be a ketone, an alcohol, an ester, an ether, an aliphatic hydrocarbon, an aromatic hydrocarbon or a halogenated hydrocarbon.

The polymerization initiator may, for example, be a peroxide or an azo compound. A chain transfer agent may be used for the polymerization.

### <Fluorinated Polymer (A2)>

The fluorinated polymer (A2) has the above units (m1) and units (hereinafter referred to also as units (m4)) derived from the following monomer (m4).

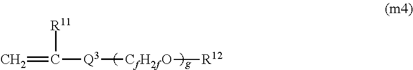

wherein R¹¹ is a hydrogen atom, a chlorine atom or a methyl group, Q³ is —COO— or —COO(CH₂)ₕ—NHCOO— (wherein h is an integer of from 1 to 4), R¹² is a hydrogen atom or —(CH₂)ᵢ—R¹³ (wherein R¹³ is a C₁₋₈ alkoxy group, a hydrogen atom, a hydroxy group or a cyano group, and i is an integer of from 1 to 25), f is an integer of from 1 to 10, and g is an integer of from 1 to 100.

### Monomer (m4):

In the formula (m4), R¹¹ is, from the viewpoint of polymerization efficiency, preferably a hydrogen atom or a methyl group, particularly preferably a methyl group. Q³ is preferably —COO—.

In a case where g is 2 or more, the plurality of (C_{f}H_{2f}O) may be the same or different. If different, their disposition may be any of random, block and alternating. In a case where f is 3 or more, C_{f}H_{2f}O may have a linear structure or a branched structure. (C_{f}H_{2f}O) may, for example, be (CH₂O), (CH₂CH₂O), (CH₂CH₂CH₂O), (CH(CH₃)CH₂O), (CH₂CH₂CH₂CH₂O).

f is preferably an integer of from 1 to 6, particularly preferably an integer of from 1 to 4, from such a viewpoint that protein is less likely to be adsorbed on the surface of the article.

g is preferably an integer of from 1 to 50, more preferably an integer of from 1 to 30, particularly preferably an integer of from 1 to 20, from such a viewpoint that an exclusion volume effect is high and protein is less likely to be adsorbed on the surface of the article.

i is preferably an integer of from 1 to 4, particularly preferably 1 or 2, from the viewpoint of excellent flexibility of the fluorinated polymer (A2).

R¹³ is preferably a hydroxy group or an alkoxy group from such a viewpoint that protein is less likely to be adsorbed on the surface of the article, particularly preferably a hydroxy group.

As the monomer (m4), a monomer (m41) represented by the following formula (m41) is preferred.

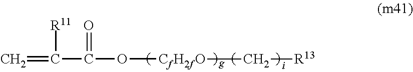

As the monomer (m4), from such a viewpoint that protein is less likely to be adsorbed on the surface of the article, the following compounds are preferred.

CH₂═CH—COO—(C₂ H₄ O)₉ —H,

CH₂═CH—COO—(C₂ H₄ O)₄ —H,

CH₂═CH—COO—(C₂ H₄ O)₅ —H,

CH₂═C(CH₃)—COO—(C₂ H₄ O)₉ —CH₃,

CH₂═CH—COO—(CH₂ O)—(C₂ H₄ O)_{g 1} —CH₂—OH,

CH₂═C(CH₃)—COO—(C₂ H₄ O)_{g 2} —(C₄ H₈ O)_{g 3} —H.

In the above compounds, g1 is an integer of from 1 to 10, g2 is from 1 to 10, and g3 is from 1 to 10.

The fluorinated polymer (A2) may have units derived from a monomer other than the monomer (m1) and the monomer (m4). As such other monomer, from the viewpoint of excellent water resistance of the surface of the article, a monomer (m5) represented by the following formula (m5) is preferred.

CH₂═CR¹⁴—COO-Q⁴-R¹⁵ (m5)

wherein R¹⁴ is a hydrogen atom, a chlorine atom or a methyl group, R¹⁵ is a C₁₋₈ alkoxy group, a hydrogen atom, a hydroxy group or a cyano group, and Q⁴ is a single bond, a C₁₋₂₀ alkylene group, a C₁₋₁₂ polyfluoroalkylene group or —CF₂—(OCF₂CF₂)_{y}—OCF₂— (wherein y is an integer of from 1 to 6).

In the formula (m5), R¹⁴ is, from the viewpoint of polymerization efficiency, preferably a hydrogen atom or a methyl group, particularly preferably a hydrogen atom.

The alkylene group and polyfluoroalkylene group for Q⁴ may be linear or branched. Q⁴ is, from the viewpoint of excellent flexibility of the fluorinated polymer (A2), preferably a C₁₋₁₂ alkylene group, particularly preferably a methylene group or an isobutylene group. R¹⁵ is, from the viewpoint of excellent water resistance, preferably a hydrogen atom.

As the monomer (m5), CH₂═CH—COO—(CH₂)₄ —H, CH₂═CH—COO(CH₂)₈ —H or CH₂═CH—COO—(CH₂)_{1 6} —H is preferred, and CH₂═CH—COO—(CH₂)₈ —H or CH₂═CH—COO—(CH₂)_{1 6} —H is particularly preferred.

In a case where the fluorinated polymer (A2) has units (m5), the units (m5) may be of one type, or of two or more types.

In a case where the fluorinated polymer (A2) has units (m5) in addition to units (m1) and units (m4), particularly preferred is a fluorinated polymer having CH₂═CHCOO(CH₂)₂ (CF₂)₅ CF₃ units, CH₂═CH—COO—(CH₂ O)—(C₂ H₄ O)_{g 1} —CH₂ —OH (g1=1 to 20) units, and CH₂═CH—COO—(CH₂)_{1 6} —H units.

In a case where the fluorinated polymer (A2) has units (m5), the proportion of units (m5) to the total of units (m1) and units (m4) is preferably from 5 to 95 mol %, particularly preferably from 10 to 90 mol %. When the proportion is at least the lower limit value of the above range, water resistance of the surface of the article will be excellent. When the proportion is at most the upper limit value of the above range, protein is less likely to be adsorbed on the surface of the article.

The fluorinated polymer (A2) may be produced in the same manner as production of the fluorinated polymer (A1) except that the monomers (m1), (m4) and (m5), are used.

In the present invention, as the fluorinated polymer (A), only one of the fluorinated polymer (A1) and the fluorinated polymer (A2) may be used, or both the fluorinated polymer (A1) and the fluorinated polymer (A2) may be used. The fluorinated polymer (A) is not limited to the above fluorinated polymer (A1) and the fluorinated polymer (A2).

### (Curable Monomer (B))

The curable monomer (B) is at least one monomer selected from the group consisting of a vinyl monomer and a cyclic ether monomer.

The vinyl monomer is preferably a vinyl monomer having no fluorine atom in its molecule, whereby the fluorinated polymer (A) is likely to be unevenly present in the vicinity of the surface of the cured product, and protein non-adsorption is likely to develop. As the vinyl monomer, a vinyl monomer having a fluorine atom(s) in its molecule may be used.

The number of polymerizable functional groups in the vinyl monomer is preferably from 1 to 20, more preferably from 1 to 10, particularly preferably from 2 to 6, from such a viewpoint that both excellent protein non-adsorption and form stability are likely to be satisfied.

The molecular weight of the vinyl monomer is preferably from 100 to 100,000, more preferably from 200 to 20,000, particularly preferably from 500 to 5,000, from such a viewpoint that both excellent protein non-adsorption and form stability are likely to be satisfied.

The vinyl monomer may be a monofunctional vinyl monomer having one polymerizable functional group, a bifunctional vinyl monomer having two polymerizable functional groups, or a multifunctional vinyl monomer having three or more polymerizable functional groups.

The monofunctional vinyl monomer may, for example, be a radical-polymerizable monomer or a cation-polymerizable monomer. The radical-polymerizable monofunctional vinyl monomer may, for example, be an alkyl (meth)acrylate (such as methyl (meth)acrylate, ethyl (meth)acrylate, butyl (meth)acrylate, hexyl (meth)acrylate, dodecyl (meth)acrylate, 2-ethylhexyl (meth)acrylate, lauryl (meth)acrylate or stearyl (meth)acrylate), benzyl (meth)acrylate, phenoxyethyl (meth)acrylate, cyclohexyl (meth)acrylate, polyethylene glycol (meth)acrylate, polypropylene glycol (meth)acrylate, 2-hydroxyethyl (meth)acrylate, 2-hydroxypropyl (meth)acrylate, N,N-dimethylaminoethyl (meth)acrylate, allyl (meth)acrylate, ethoxyethyl (meth)acrylate, methoxyethyl (meth)acrylate, styrene, methylstyrene, chloromethylstyrene, vinyl acetate, vinyl propionate, N-vinylpyrrolidone, N,N-dimethylacrylamide, tris(trimethylsiloxysilyl)propyl vinyl carbamate, (trimethoxysiloxy)silylpropyl methacrylate, (3-methacryloyloxy-2-hydroxypropyloyloxy)propylbis(trimethoxysiloxy)methylsilane or methyl di(trimethylsiloxy)silyl propyl glycerol methacrylate. The cation-polymerizable monofunctional vinyl monomer may, for example, be an alkyl vinyl ether (such as cyclohexylmethyl vinyl ether, isobutyl vinyl ether, cyclohexyl vinyl ether or ethyl vinyl ether) or 4-hydroxybutyl vinyl ether.

As the monofunctional vinyl monomer, the following compounds may also be mentioned.

CH₂═CHO(CH₂)₃ COOCH₃,

CH₂═CHO(CH₂)₃ CH₂ OH,

CH₂═CHCOO—(C₂ H₄ O)₂ —CH₃,

CH₂═CHCOO—(C₂ H₄ O)₄ —CH₃,

CH₂═C(CH₃)COO—(C₂ H₄ O)₂ —CH₃,

CH₂═C(CH₃)COO—(C₂ H₄ O)₄ —CH₃, etc.

The bifunctional vinyl monomer may, for example, be a diene (such as norbornadiene, butadiene or 1,4-pentadiene), bisphenol A glycidyl di(meth)acrylate, propoxylated ethoxylated bisphenol A di(meth)acrylate, 9,9-bis[4-(2-(meth)acryloyloxyethoxy)phenyl]fluorene, ethoxylated bisphenol A di(meth)acrylate, 9,9-bis[4-2-(meth)acryloyloxy ethoxy)phenyl]fluorene, propoxylated bisphenol A di(meth)acrylate, tricyclodecane dimethanol di(meth)acrylate, 1,10-decanediol di(meth)acrylate, 1,6-hexanediol di(meth)acrylate, 1,9-nonanediol di(meth)acrylate, 1,3-butanediol diacrylate, dipropylene glycol di(meth)acrylate, tripropylene glycol di(meth)acrylate, ethylene glycol di(meth)acrylate or polyethylene glycol di(meth)acrylate.

Further, as the bifunctional vinyl monomer, the following compounds may also be mentioned.

CH₂═CHOCH₂ CH═CH₂,

CH₂═CHOCH₂ CH₂CH═CH₂,

CH₂═CHOCH(CH₃)CH₂ CH═CH₂,

CH₂═CHOCH₂ OCH═CH₂,

CH₂═CHCH₂ C(OH)(CH₃)CH₂ CH═CH₂,

CH₂═CHCH₂ C(OH)(CH₃)CH═CH₂,

CH₂═CHCOO—(C₂ H₄ O)₂ —COCH═CH₂

CH₂═CHCOO—(C₂ H₄ O)₄ —COCH═CH₂,

CH₂═CHCOO—CH₂ CH(OH)CH₂ —OCOC(CH₃)═CH₂, etc.

The multifunctional vinyl monomer may, for example, be ethoxylated isocyanuric tri(meth)acrylate, £-caprolactone modified tris-(2-(meth)acryloxyethyl)isocyanurate, pentaerythritol tri(meth)acrylate, trimethylolpropane tri(meth)acrylate, ditrimethylolpropane tetra(meth)acrylate, ethoxylated pentaerythritol tetra(meth)acrylate, pentaerythritol tetra(meth)acrylate, dipentaerythritol poly(meth)acrylate or dipentaerythritol hexa(meth)acrylate.

The vinyl monomer is preferably radical polymerizable in view of high reactivity. The vinyl monomer is preferably a bifunctional vinyl monomer or a multifunctional vinyl monomer in view of high solvent resistance after crosslinking, and is particularly preferably a bifunctional vinyl monomer or a multifunctional vinyl monomer having at most 6 polymerizable functional groups in view of small shrinkage on curing.

The vinyl monomer is, from such a viewpoint that both protein non-adsorption and form stability are likely to be satisfied, preferably an alkyl (meth)acrylate, bisphenol A glycidyl di(meth)acrylate, trimethylolpropane tri(meth)acrylate or polyethylene glycol di(meth)acrylate, more preferably an alkyl (meth)acrylate, bisphenol A glycidyl di(meth)acrylate or trimethylolpropane tri(meth)acrylate. The vinyl monomer may be of one type, or of two or more types.

The cyclic ether monomer is preferably a cyclic ether monomer having no fluorine atom in its molecule from such a viewpoint that the fluorinated polymer (A) is likely to be unevenly present in the vicinity of the surface of the cured product, and protein non-adsorption is likely to develop. As the cyclic ether monomer, a vinyl monomer having a fluorine atom(s) in its molecule may be used.

The number of polymerizable functional groups in the cyclic ether monomer is preferably from 1 to 20, more preferably from 1 to 10, particularly preferably from 2 to 6, from such a viewpoint that both excellent protein non-adsorption and form stability are likely to be satisfied.

The molecular weight of the cyclic ether monomer is preferably from 50 to 50,000, more preferably from 100 to 10,000, particularly preferably from 100 to 5,000, from such a viewpoint that excellent protein non-adsorption and form stability are likely to be satisfied.

The cyclic ether monomer may be a monofunctional the cyclic ether monomer having one polymerizable functional group, a cyclic ether monomer having two polymerizable functional groups, or a cyclic ether monomer having three or more polymerizable functional groups.

The monofunctional the cyclic ether monomer may, for example, be ethylene oxide, propylene oxide, 1,3-butylene oxide, ethyl glycidyl ether, propyl glycidyl ether, butyl glycidyl ether, 3-ethyl-3-hydroxymethyloxetane, 3-ethyl-3-allyloxymethyloxetane, 3-ethyl-3-methallyloxymethyloxetane or tetrahydrofuran.

The bifunctional cyclic ether monomer may, for example, be bisphenol A diglycidyl ether.

The multifunctional cyclic ether monomer may, for example, be tris-(2,3-epoxypropyl) isocyanurate, tris-(3,4-epoxybutyl) isocyanurate, tris-(4,5-epoxypentyl) isocyanurate, tris-(5,6-epoxyhexyl) isocyanurate or tris(glycidyloxyethyl) isocyanurate.

The cyclic ether monomer is preferably a bifunctional cyclic ether monomer or a multifunctional cyclic ether monomer in view of high solvent resistance after crosslinking, and particularly preferably a bifunctional cyclic ether monomer or a multifunctional cyclic ether monomer having at most 6 polymerizable functional groups in view of small shrinkage on curing.

The cyclic ether monomer is, from such a viewpoint that both protein non-adsorption and form stability are likely to be satisfied, preferably 1,3-butylene oxide, butyl glycidyl ether, bisphenol A diglycidyl ether or 3-ethyl-3-hydroxymethyloxetane, more preferably 1,3-butylene oxide, butyl glycidyl ether or bisphenol A diglycidyl ether. The cyclic ether monomer may be of one type, or of two or more types.

The protein adhesion inhibitor of the present invention may contain only a vinyl monomer, may contain only a cyclic ether monomer, or may contain both vinyl monomer and cyclic ether monomer, as the curable monomer. The protein adhesion inhibitor of the present invention preferably contains, as the curable monomer, only a vinyl monomer or only a cyclic ether monomer.

### (Polymerization Initiator)

The protein adhesion inhibitor of the present invention preferably contains a polymerization initiator, particularly preferably contains a photopolymerization initiator. The photopolymerization initiator is to cause radical reaction or ionic reaction by light, and it is preferably a photopolymerization initiator which causes radical reaction.

As the photopolymerization initiator, a known photopolymerization initiator may be used. Specifically, for example, an acetophenone initiator (such as acetophenone, p-tert-butyltrichloroacetophenone or chloroacetophenone), a benzoin initiator (such as benzyl, benzoin, benzoin methyl ether or benzoin ethyl ether), a benzophenone initiator (such as benzophenone, benzoylbenzoic acid or methyl benzoylbenzoate), a thioxanthone initiator (such as thioxanthone, 2-chlorothioxanthone or 2-methylthioxanthone), or perfluoro(tert-butylperoxide), perfluorobenzoyl peroxide, etc., each having fluorine atoms, may be mentioned. Further, α-acyloxime ester, benzyl-(o-ethoxycarbonyl)-α-monooxime, acylphosphine oxide, glyoxy ester, 3-ketocoumarin, 2-ethylanthraquinone, cam phorquinone, tetramethylthiuram sulfide, azobisisobutyronitrile, benzoyl peroxide, dialkyl peroxide or tert-butyl peroxypivarate may also be used. The photopolymerization initiator may be used alone or in combination of two or more.

### (Other Component)

The protein adhesion inhibitor of the present invention may contain, as the case requires, a component other than the fluorinated polymer (A), the curable monomer (B) and the polymerization initiator. Such other component may, for example, be a photosensitizer or a leveling agent.

### (Proportion of Components)

The content of the fluorinated polymer (A) is preferably from 0.01 to 50.00 mass %, more preferably from 0.01 to 10.00 mass %, particularly preferably from 0.1 to 10.00 mass % to the total mass of the protein adhesion inhibitor. When the content is at least the lower limit value of the above range, protein is hardly attached to the surface of the article. When the content is at most the upper limit value of the above range, the article will be excellent in mechanical strength.

The content of the curable monomer (B) is preferably from 50.00 to 99.99 mass %, more preferably from 90.00 to 99.98 mass %, particularly preferably from 90.00 to 99.90 mass % to the total mass of the protein adhesion inhibitor. When the content is at least the lower limit value of the above range, the article will be excellent in mechanical strength. When the content is at most the upper limit value of the above range, physical properties reflecting the charge composition will be obtained.

In a case where the protein adhesion inhibitor of the present invention contains a polymerization initiator, the content of the polymerization initiator is preferably from 0.01 to 5.00 mass %, more preferably from 0.01 to 3.00 mass %, particularly preferably from 0.10 to 3.00 mass % to the total mass of the protein adhesion inhibitor. When the content is at least the lower limit value of the above range, curing will sufficiently proceed. When the content is at most the upper limit value of the above range, the molecular weight of the cured product will be sufficiently high.

### (Coefficient α)

The present inventors have conducted studies on the form stability of a cured product of the protein adhesion inhibitor and as a result, found that the coefficient α represented by the following formula (I) relates to the degree of polymerization shrinkage of the protein adhesion inhibitor, the polymerization shrinkage becomes small by decreasing the coefficient α, and the form stability of the cured product improves.

In the protein adhesion inhibitor of the present invention, the coefficient α represented by the following formula (I) is at most 10, whereby the polymerization shrinkage becomes small, and the form stability of the obtainable cured product will be excellent. Accordingly, for example, in a case where a film-form cured product (hereinafter sometimes referred to as a “cured film”) is to be formed from the protein adhesion inhibitor of the present invention, the obtainable cured film is hardly warp, and can easily be bonded to the surface of a substrate such as a cell culture vessel or a plate.

The coefficient α of the protein adhesion inhibitor of the present invention is preferably at least 1, whereby the protein adhesion inhibitor can readily be formed into a film. The coefficient α is more preferably from 1 to 10, further preferably from 5 to 8.

$$α = ( \frac{1000}{{M}_{1}} \times {N}_{1} \times \frac{{W}_{1}}{100} ) \times 2 + ( \frac{1000}{{M}_{2}} \times {N}_{2} \times \frac{{W}_{2}}{100} ) \qquad ( I )$$

wherein M₁ is the molecular weight of the vinyl monomer, N₁ is the number of polymerizable functional groups in the vinyl monomer, W₁ is the content (mass %) of the vinyl monomer to the total mass of the protein adhesion inhibitor, M₂ is the molecular weight of the cyclic ether monomer, N₂ is the number of polymerizable functional groups in the cyclic ether monomer, and W₂ is the content (mass %) of the cyclic ether monomer to the total mass of the protein adhesion inhibitor.

In a case where the protein adhesion inhibitor of the present invention contains two or more types of vinyl monomers, M₁ is a mass-averaged value of the molecular weights of the respective vinyl monomers, N₁ is the mass-averaged value of the numbers of polymerizable functional groups in the respective vinyl monomers, and W₁ is the total content of the vinyl monomers. Likewise, in a case where the protein adhesion inhibitor of the present invention contains two or more types of cyclic ether monomers, M₂ is the mass-averaged value of the molecular weights of the respective cyclic ether monomers, N₂ is the mass-averaged value of the numbers of polymerizable functional groups in the respective cyclic ether monomers, and W₂ is the total content of the cyclic ether monomers.

### (Application)

As an application of the protein adhesion inhibitor of the present invention, a medical device is particularly effective.

If protein is adsorbed on the surface of a medical device, cells are further attached to the adsorbed protein. Therefore, by inhibiting protein adsorption, cell adhesion can also be inhibited. Accordingly, the protein adsorption inhibitor of the present invention may be used, for example, to prevent cell adhesion, that is, as a cell adhesion inhibitor. A “cell” is the most fundamental unit constituting a living body and means one which has, in the interior of the cell membrane, the cytoplasm and various organelles. Nuclei containing DNA may be contained or may not be contained inside the cell.

Animal-derived cells include germ cells (sperm, ova, etc.), somatic cells constituting a living body, stem cells, progenitor cells, cancer cells separated from a living body, cells (cell line) which are separated from a living body and have won immortalized ability and thus are stably maintained outside the body, cells separated from a living body and artificially genetically engineered, cells separated from a living body and having nuclei artificially replaced, etc.

Somatic cells constituting a living body include fibroblasts, bone marrow cells, B lymphocytes, T lymphocytes, neutrophils, erythrocytes, platelets, macrophages, monocytes, bone cells, bone marrow cells, pericytes, dendritic cells, keratinocytes, fat cells, mesenchymal cells, epithelial cells, epidermal cells, endothelial cells, vascular endothelial cells, hepatocytes, cartilage cells, cumulus cells, neural cells, glial cells, neurons, oligodendrocytes, microglia, astrocytes, cardiac cells, esophagus cells, muscle cells (for example, smooth muscle cells, skeletal muscle cells), pancreatic beta cells, melanin cells, hematopoietic progenitor cells, mononuclear cells, etc.

The somatic cells include cells taken from optional tissues, such as skin, kidneys, spleen, adrenal gland, liver, lung, ovary, pancreas, uterus, stomach, colon, small intestine, large intestine, bladder, prostate, testis, thymus, muscle, connective tissue, bone, cartilage, vascular tissue, blood, heart, eye, brain, nervous tissue, etc.

The stem cells are cells having both an ability to replicate themselves and an ability to be differentiated into cells of other multiple systems, and include embryonic stem cells (ES cells), embryonic carcinoma cells, embryonic germ stem cells, induced pluripotent stem cells (iPS cells), neural stem cells, hematopoietic stem cells, mesenchymal stem cells, liver stem cells, pancreatic stem cells, muscle stem cells, germ stem cells, intestinal stem cells, cancer stem cells, hair follicle stem cells, etc.

The progenitor cells are cells at an intermediate stage during differentiation into specific somatic or germ cells from the stem cells.

The cancer cells are cells that have acquired an unlimited proliferative capacity as derived from somatic cells.

A cell line is cells which have acquired an unlimited proliferative capacity by an artificial manipulation in vitro, and includes HCT116, Huh7, HEK293 (human embryonic kidney cells), HeLa (human cervical carcinoma cell line), HepG2 (human liver cancer cell line), UT7/TPO (human leukemia cell line), CHO (Chinese hamster ovary cell line), MDCK, MDBK, BHK, C-33A, HT-29, AE-1, 3D9, Ns0/1, Jurkat, NIH3T3, PC12, S2, Sf9, Sf21, High Five, Vero, etc.

Further, the protein adhesion inhibitor of the present invention may also be used for marine structures such as ships, bridges, marine tanks, port facilities, submarine bases, offshore oil drilling equipment, etc. By applying the protein adhesion inhibitor of the present invention to a marine structure, it is possible to prevent adhesion of protein to the marine structure. As a result, it is possible to prevent adhesion of aquatic organisms such as shellfish (barnacles, etc.), seaweeds (green laver, sea lettuce, etc.), etc.

As described above, the protein adhesion inhibitor of the present invention is a composition comprising the fluorinated polymer (A) and the curable monomer (B), and can be cured to form a cured product. Since the fluorinated polymer (A) has low surface tension, it is unevenly present in the vicinity of the surface of the cured product. Accordingly, at least one of the groups (1) to (3) in the fluorinated polymer (A) is disposed on the surface of the cured product, and excellent protein non-adsorption will develop.

Further, in the protein adhesion inhibitor of the present invention, the type and the content of the curable monomer (B) are controlled so that the coefficient α represented by the formula (I) is at most 10. Accordingly, the shrinkage on curing of the protein adhesion inhibitor becomes small, and the obtainable cured product has excellent form stability.

### [Cured Product]

The cured product of the present invention is a cured product formed by curing the protein adhesion inhibitor of the present invention. The shape of the cured product is not particularly limited and may properly be determined depending upon the application, and may, for example, be film form. By bonding the cured film to the surface of a substrate such as a cell culture vessel or a plate, adsorption of proteins on the surface can be inhibited. The shape of the cured product itself may be the shape of a device such as a cell culture vessel.

On the surface of the cured product of the present invention, surface microfabrication may be formed, for example, a convexo-concave pattern or a line-and-space pattern may be applied. The convexo-concave pattern may, for example, be a pattern having a plurality of wells regularly formed.

The method for producing a cured product of the present invention may be a method of obtaining a formed product by a known forming method by using the protein adhesion inhibitor or a coating solution containing the protein adhesion inhibitor, followed by curing reaction by irradiation with light. The forming method is not particularly limited and may, for example, be pressing of a mold, injection molding, extrusion, forming by a 3D printer, or cast molding. In the case of a cured film having a convexo-concave pattern on its surface, the after-described production method is preferably employed.

### [Coating Solution]

In a case where the protein adhesion inhibitor of the present invention is liquid at room temperature (20 to 25° C.), the protein adhesion inhibitor may be used as it is as a coating solution. In a case where the viscosity of the protein adhesion inhibitor of the present invention is not sufficiently low at room temperature, the protein adhesion inhibitor may be mixed with a solvent to obtain a coating solution.

The solvent may be a non-fluorinated solvent or a fluorinated solvent. The non-fluorinated solvent may, for example, be an alcohol solvent or a halogenated solvent. The solvent may, for example, be specifically ethanol, methanol, acetone, chloroform, ASAHIKLIN AK225 or AC6000 (registered trademark by Asahi Glass Company, Limited). The solvent may be used alone or in combination of two or more.

In a case where a solvent is used, the concentration of the protein adhesion inhibitor in the coating solution is preferably from 0.001 to 10.00 mass %, particularly preferably from 0.01 to 5.00 mass %. When the concentration is within the above range, the coating solution may be uniformly applied, and a uniform film may be formed.

### [Method for Producing Cured Product]

The method for producing a cured product of the present invention is typically a method for producing a cured film having a convexo-concave pattern on its surface. By the method for producing a cured product of the present invention, a coating film formed from a coating solution containing the protein adhesion inhibitor is irradiated with light in such a state that a convexo-concave plane formed on the surface of a mold is pressed against the coating film, thereby to cure the coating film to obtain a cured film. In such a manner, a cured film having a convexo-concave pattern complementary to the convexo-concave plane of the mold transcribed on its surface is obtained.

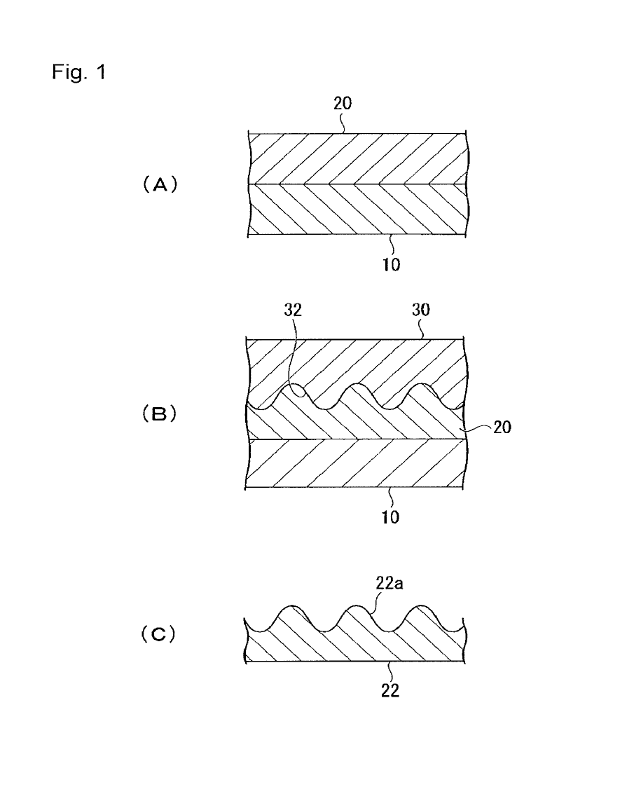

Now, an example of the production method of the present invention will be described with reference to FIG. 1. In FIG. 1, a substrate sheet, a coating film and the like are illustrated in their schematic cross sections.

As shown in FIG. 1(A), a coating solution is applied to a substrate sheet 10 to form a coating film 20. Then, as shown in FIG. 1(B), the coating film 20 is irradiated with light in such a state that a convexo-concave plane 32 of a mold 30 is pressed against the coating film 20 to cure the coating film 20. After curing, the substrate sheet 10 and the mold 30 are removed to obtain a cured film 22 as shown in FIG. 1(C). On the surface of the obtained cured film 22, a convexo-concave pattern 22a complementary to the convexo-concave plane 32 of the mold 30 is formed. The thickness of the cured film is preferably from 1.0 μm to 5.0 mm, particularly preferably from 1.0 μm to 1.0 mm.

The method for applying the coating solution may be a known wet coating method and may, for example, be a method of using a coating apparatus such as a brush, a roller, a dip coater, a spray coater, a roll coater, a die coater, an applicator or a spin coater. The thickness of the coating film is preferably from 1.0 μm to 5.0 mm, particularly preferably from 1.0 μm to 1.0 mm.

The light irradiation method may be a known method.

According to the above-described method for producing a cured product of the present invention, it is possible to obtain a cured film having excellent protein non-adsorption and excellent form stability as well, and having warpage suppressed.

Further, according to the method for producing a cured product of the present invention, since the volume shrinkage of the protein adhesion inhibitor is small, the transcription accuracy of the convexo-concave plane of the mold is high. Accordingly, a convexo-concave pattern having dimensions and form highly accurately controlled, can be formed even by surface microfabrication such as a case of forming a plurality of minute wells on the surface of the cured product, as compared with conventional laser processing. Particularly a cured film having minute wells having uniform aperture and depth formed on its surface, obtained by the present invention, is preferred in that cells cultured in the respective wells will be uniform in size.

### [Article]

The article of the present invention comprises on at least a part of its surfaces the cured product of the protein adhesion inhibitor of the present invention, whereby adsorption of proteins and attachment of cells on the surface of the article can be inhibited. The article of the present invention is preferably a medical device.

Specific examples of the medical device may, for example, be pharmaceuticals, quasi-drugs, medical tools, etc. The medical tools are not particularly limited and may, for example, be cell culture vessels, cell culture sheets, cell trapping filters, vials, plastic-coated vials, syringes, plastic-coated syringes, ampoules, plastic coated ampoules, cartridges, bottles, plastic-coated bottles, pouches, pumps, sprayers, plugs, plungers, caps, lids, needles, stents, catheters, implants, contact lenses, micro-channel chips, drug delivery system materials, artificial blood vessels, artificial organs, blood dialysis membranes, guard wires, blood filters, blood storage packs, endoscopes, bio-chips, sugar chain synthesis equipment, molding auxiliary materials, packaging materials, etc. Among them, cell culture vessels, cell culture sheets, cell trapping filters and micro-channel chips are preferred. The article of the present invention which is a cell culture vessel or a cell culture sheet, is useful in the regenerative medicine field since excellent cell proliferation capacity is obtainable, and more efficient mass cell culture will be possible.

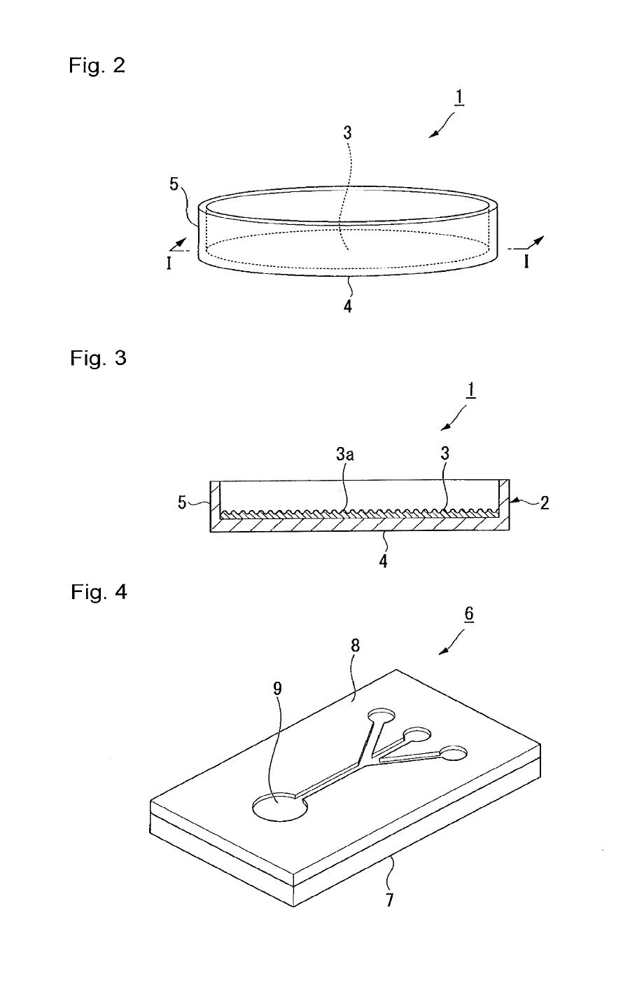

The article of the present invention preferably comprises a substrate and a coating layer formed of a cured product of the protein adhesion inhibitor of the present invention on the substrate. As a specific example of the article according to such an embodiment, a medical device 1 illustrating in FIGS. 2 and 3 may be mentioned. The medical device 1 is a petri dish which is one of cell culture vessels. The medical device 1 comprises a substrate 2 and a coating layer 3 formed on the substrate 2.

The substrate 2 comprises a bottom surface portion 4 having a circular shape in plan view, and a side surface portion 5 which rises along the entire circumference of the peripheral edge of the bottom surface portion 4, and has a shape of a container with the top being open.

The coating layer 3 is formed of the cured product of the protein adhesion inhibitor of the present invention and is formed on the top surface of the bottom surface portion 4 of the substrate 2. On the surface of the coating layer 3 in this example, a convexo-concave pattern 3a is formed. The coating layer 3 may be formed, for example, by bonding the cured film 22 obtained by the above production method to the top surface of the bottom surface portion 4 of the substrate 2.

Further, as an article comprising a substrate and a coating layer formed on the substrate, a medical device 6 shown in FIG. 4 may also be mentioned. The medical device 6 is a micro-channel chip. The medical device 6 comprises a planar substrate 7 and a coating layer 8 formed on the substrate 7.

On the surface of the coating layer 8, a wetted part 9 such as a channel, formed of a concave portion, is formed. The coating layer 8 may be formed, for example, by bonding on the top surface of the substrate 7, a cured film obtained by curing a coating film formed from a coating solution by using a mold having a convexo-concave plane having a convex portion complementary to the wetted part 9 formed, in such a state that the convexo-concave plane is pressed against the coating film.

The material constituting the substrate in the article is not particularly limited and may, for example, be a resin such as polyethylene terephthalate, polystyrene, polycarbonate, polypropylene or a tetrafluoroethylene/ethylene copolymer (ETFE), or glass. Usually, from the viewpoint of the material cost and processing cost, a resin is preferred. On the other hand, for an application e.g. to be used for high accuracy analysis, preferred is glass which is highly transparent as the material itself, which emits less fluorescence, which is chemically stable and which is excellent in rigidity.

The thickness of the coating layer is preferably from 100 nm to 10,000 μm, particularly preferably from 100 nm to 1,000 μm. When the thickness of the coating layer is at least the above lower limit value, protein is less likely to be adsorbed. When the thickness of the coating layer is at most the above upper limit value, the coating layer tends to intimately adhere to the surface of the substrate constituting the device.

The method of bonding the coating layer to the substrate is not particularly limited. One which exhibits sufficient adhesion to both the coating layer and the substrate may suitably be used, and for example, a cyanoacrylate adhesive, a silicone-modified acrylic adhesive or an epoxy-modified silicone adhesive may, for example, be mentioned. For example, in a case where polystyrene is used as a material for forming the substrate, a cyanoacrylate adhesive is used.

The above-described article of the present invention has excellent protein non-adsorption, and is excellent in the form stability of the coating layer, and accordingly drawbacks such as peeling are less likely to occur.

Further, the article of the present invention is not limited to one comprising a substrate and a coating layer, and may be formed solely of a cured product of the protein adhesion inhibitor of the present invention.

### EXAMPLES

Now, the present invention will be described in detail with reference to Examples, but the present invention is not limited by the following description. Ex. 1 to 5 and 10 to 14 are Examples of the present invention, and Ex. 6 to 9 and 15 to 17 are Comparative Examples.

### [Evaluation Methods]

### (Copolymer Composition)

20 mg of a non-polymerizable fluorinated polymer (A) was dissolved in chloroform, and the copolymer composition was determined by ¹H-NMR.

### (Fluorine Atom Content Q_{F})

The fluorine atom content Q_{F} of a non-polymerizable fluorinated polymer (A) was determined in accordance with the formula: Q_{F}=[19×N_{F}/M_{A}]×100 from N_{F} and M_{A} calculated based on measurement by ¹H-NMR, ion chromatography and elemental analysis.

### (Glass Transition Temperature (Tg))

The glass transition temperature of a non-polymerizable fluorinated polymer (A) was measured by raising or lowering the temperature between −30° C. to 200° C. at a rate of 10° C./min. by DSC (manufactured by TA Instruments). The temperature for a change from the rubber state to the glass state in the second cycle of the temperature lowering, was adopted as the glass transition temperature.

### [Molecular Weight]

The number average molecular weight (Mn), mass average molecular weight (Mw) and molecular weight distribution (Mw/Mn) of a fluorinated polymer, were measured by means of a GPC device (HLC8220, manufactured by Tosoh Corporation) using tetrahydrofuran as a solvent.

### (Protein Non-Adsorption)

Protein non-adsorption was determined in accordance with the following procedure (1) to (7).

(1) Preparation of Coloring Solution and Protein Solution

As the coloring solution, one having 50 mL of a peroxidase color solution (3,3′,5,5′-tetramethylbenzidine (TMBZ), manufactured by KPL, Inc.) and 50 mL of TMB Peroxidase Substrate (manufactured by KPL, Inc.) mixed, was used.

As the protein solution, one having protein (POD-goat anti mouse IgG, manufactured by Bio-Rad Laboratories, Inc.) diluted to 16,000-fold with phosphate buffered saline (D-PBS, manufactured by Sigma Co.), was used.

(2) Preparation of Cured Product

99.0 g of ethanol was added to 1.0 g of the protein adhesion inhibitor composition obtained in each Ex. to prepare a coating solution. The coating solution was applied to a polymethyl methacrylate (PMMA) sheet to form a coating film having a thickness of 10.0 μm. Then, a convexo-concave plane comprising minute irregularities formed on the surface of a mold was pressed against the coating film, and in such a state, UV irradiation was conducted under conditions of 3,000 mJ/cm² in a nitrogen atmosphere to obtain a cured film having a plurality of minute wells formed on its surface.

(3) Protein Adsorption

To 3 wells of the cured film, 2 mL of the protein solution was dispensed (using 2 mL per well) and left to stand at room temperature for one hour.

As a blank, the protein solution was dispensed to 3 wells of a 96-well microplate in an amount of 2 μL (using 2 μL per well).

(4) Washing of Wells

Then, each of the wells to which the protein solution was dispensed of the cured film was washed four times with 4 mL of phosphate buffered saline (D-PBS, manufactured by Sigma Co.) having 0.05 mass % of a surfactant (Tween 20, manufactured by Wako Pure Chemical Industries, Ltd.) incorporated (using 4 mL per well).

(5) Dispensing of Coloring Solution

Then, to each of the washed wells of the cured film, 2 mL of the coloring solution was dispensed (using 2 mL per well), and a coloring reaction was carried out for 7 minutes. The coloring reaction was stopped by adding 1 mL of 2N sulfuric acid (using 1 mL per well).

As the blank, to the 96-well microplate, 100 μL of the coloring solution was dispensed (using 100 μL per well), and a coloring reaction was carried out for 7 minutes. The coloring reaction was stopped by adding 50 μL of 2N sulfuric acid (using 50 μL per well).

(6) Preparation for Measurement of Absorbance

Then, from each of the wells of the cured film, 150 μL of the liquid was taken and transferred to the 96-well microplate.

(7) Measurement of Absorbance and Protein Adsorption Rate W

As to the absorbance, the absorbance at 450 nm was measured by MTP-810Lab (manufactured by Corona Electric Co., Ltd.). Here, the average value of the absorbance (N=3) of the blank was designated as A₀. The absorbance of the liquid transferred from 3 wells of the cured film to the 96-well microplates was designated as A₁.

With respect to each absorbance A₁, the protein adsorption rate P₁ (%) was obtained by the following formula, and the protein adsorption rate P was set to be the average value.

P₁=(A₁/(A₀×(100/dispensed amount of the protein solution in the blank))×100=(A₁/(A₀×(100/2 μL)))×100

(Evaluation Standards)

The protein non-adsorption was evaluated on the basis of the following standards.

◯ (good): The protein adsorption rate P being at most 0.2%.

x (bad): The protein adsorption rate P being higher than 0.2%.

(Evaluation of Cytotoxicity)

From the cured film in each Ex. obtained in “(2) preparation of cured product” in the above (protein non-adsorption), 24 circular test pieces having a diameter of 14 mm were prepared. Each of the test pieces was kept at rest on each of a 24-well microplate, 0.5 mL of a 10% FBS⋅EMEM medium was put in each well to immerse the test piece overnight, and the medium was taken as a test sample culture medium. The 10% FBS-EMEM means an E-MEM (Earle-minimum essential medium) medium having 10% of FBS (fetal bovine serum) added and having set to equilibrate under 5% carbon dioxide gas.

Further, myeloma cells were adjusted with the 10% FBS-EMEM to achieve 100 cells/mL, which were added to each of wells of a 24-well microplate in an amount of 0.5 mL/well and cultured in an incubator for 4 hours. Then, to each well from which the entire medium was withdrawn and which contained only myeloma cells, 0.5 mL of the test sample culture medium in each Ex. was added, followed by culture for one day. Quantitative measurement of the proliferation rate of myeloma cells by this culture was conducted by alamarBlue assay.

Cytotoxicity was evaluated on the basis of the following standards, where the proliferation rate of an example (control) in which myeloma cells were cultured only in a 10% FBS⋅EMEM medium, instead of the test sample culture medium in each Ex., under the same conditions, was 100%.

◯ (good): The cell proliferation rate being at least 80%.

x (bad): The cell proliferation rate being less than 80%.

### (Form Stability)

99.0 g of ethanol was added to 1.0 g of the protein adhesion inhibitor composition obtained in each Ex. to prepare a coating solution. The coating solution was applied to the entire surface of one side of a PMMA sheet of 5 cm×5 cm×300 μm in thickness to form a coating film having a thickness of 10 μm. Then, UV irradiation was conducted under conditions of 3,000 mJ/cm² in a nitrogen atmosphere to cure the coating film. The sheet after curing was placed on a plane so that the cured film faced upward, and the warpage was measured. The warpage is a distance (mm) between the plane and a portion farthest from the plane at the circumference of the sheet after curing. The form stability was evaluated on the basis of the following standards.

◯ (good): The warpage being at most 1 mm.

x (bad): The warpage being more than 1 mm.

### [Raw Materials Used]

The raw materials used in Examples are shown below.

### (Monomer Used for Preparation of Fluorinated Polymer (A))

C6FA: CH₂═CHCOO(CH₂)₂ (CF₂)₅ CF₃

2-EHA: 2-ethylhexyl acrylate (CH₂═CHCOOCH₂ CH(C₂ H₅)CH₂ CH₂ CH₂ CH₃).

PEG9A: polyethylene glycol monoacrylate (EO number (average): 9) (CH₂═CHCOO(C₂ H₄ O)₉ H).

MPC: 2-methacryloyloxyethyl phosphorylcholine (CH₂═C(CH₃)COO((CH₂)₂ OPO⁻ (CH₂)₂ N⁺ (CH₃)₃).

### (Curable Monomer (B))

Monomer (B-11): methyl methacrylate (number of polymerizable functional group N₁: 1, molecular weight M₁:100).

Monomer (B-12): hexyl methacrylate (number of polymerizable functional group N₁: 1, molecular weight M₁:170).

Monomer (B-13): dodecyl methacrylate (number of polymerizable functional group N₁: 1, molecular weight M₁:254).

Monomer (B-14): bisphenol A glycidyl dimethacrylate (number of polymerizable functional group N₁: 2, molecular weight M₁: 513).

Monomer (B-21): 1,2-butylene oxide (number of polymerizable functional group N₂: 1, molecular weight M₂: 72).

Monomer (B-22): butyl glycidyl ether (number of polymerizable functional group N₂:1, molecular weight M₂:130).

Monomer (B-23): bisphenol A diglycidyl ether (number of polymerizable functional group N₂:2, molecular weight M₂:340).

### (Polymerization Initiator)

V-601: tradename “V-601” (oil-soluble azo polymerization initiator, manufactured by Wako Pure Chemical Industries, Ltd.).

IC907: tradename “IRGACURE 907” (photopolymerization initiator, manufactured by BASF).

TTHFP: tri-p-tolylsulfonium hexafluorophosphate (photopolymerization initiator, manufactured by Tokyo Chemical Industry Co., Ltd.).

AIBN: azobisisobutyronitrile (photopolymerization initiator, manufactured by Tokyo Chemical Industry Co., Ltd.).

### Production Example 1

In a 100 mL pressure-resistant glass bottle, 40 g of 2-EHA, 40 g of PEG9A, 0.66 g of V-601 and 49.8 g of m-xylene hexafluoride (manufactured by Central Glass Co., Ltd., hereinafter sometimes referred to as “m-XHF”) were charged, and then, sealed and heated for 16 hours at 70° C. to obtain a reaction solution. To this reaction solution, 20 g of C6FA, 40 g of m-XHF and 0.48 g of V-601 were charged, and then, sealed and heated for 16 hours at 70° C., to obtain fluorinated polymer (A-1). The copolymer composition of the obtained fluorinated polymer (A-1) was measured and as a result, the copolymer was confirmed to have PEG9A units, C6FA units and 2-EHA units in a molar ratio of 24:14:62 (mass ratio of 40:20:40). Further, of the fluorinated polymer (A-1), the number average molecular weight (Mn) was 17,000, the mass average molecular weight (Mw) was 40,000, the molecular weight distribution (mass average molecular weight (Mw)/number average molecular weight (Mn)) was 2.3, the fluorine atom content Q_{F} was 11.8 ass %, and the glass transition temperature was 10° C.

### Production Example 2

0.886 g (3.0 mmol) of MPC and 3.025 g (7.0 mmol) of C6FMA were weighed into a 300 mL three-necked flask, and 0.391 g of AIBN as a polymerization initiator, and 15.6 g of ethanol as a polymerization solvent were added to obtain a solution. The charge molar ratio of C6FMA to MPC was made to be C6FMA/MPC=70/30, the total concentration of the monomers in the solution was made to be 20 mass %, and the initiator concentration was made to be 1 mass %.

Inside of the flask containing the solution was thoroughly purged with argon, and the flask was sealed and heated for 16 hours at 75° C. to conduct a polymerization reaction. The reaction solution was cooled with ice and then, dropped in diethyl ether, to precipitate a polymer. The obtained polymer was sufficiently washed with diethyl ether, and then dried under reduced pressure to obtain white powdery fluorinated polymer (A-2).

The copolymer composition of the obtained fluorinated polymer (A-2) was obtained and found to be C6FMA units/MPC units=44/56 (molar ratio). Of the fluorinated polymer (A-2), the fluorine atom content Q_{F} was 30.6 mass %, and the glass transition temperature was 117° C.

### Ex.1

Monomer (B-12) as the curable monomer (B), I-907 as the photopolymerization initiator, and the fluorinated polymer (A-1) were mixed so that their mass ratio would be 77:3:20 to prepare a protein adhesion inhibitor.

### Ex.2 to 17

Each protein adhesion inhibitor was prepared in the same manner as in Ex. 1 except that the composition was changed as identified in Table 1 or 2.

The composition and evaluation results of the protein adhesion inhibitor in each Ex. are shown in Table 1.

TABLE 1

| | | | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|---|---|
| Fluorinated | | Type | A-1 | A-1 | A-1 | A-1 | A-1 | A-1 | A-1 | A-1 | A-1 |
| polymer (A) | | Fluorine atom content Q_{F} [mass %] | 11.8 | 11.8 | 11.8 | 11.8 | 11.8 | 11.8 | 11.8 | 11.8 | 11.8 |
| | | Content [mass %] | 20 | 10 | 10 | 10 | 10 | 10 | 10 | — | 10 |
| Curable | Vinyl | Type | B-12 | B-13 | B-14 | — | — | B-11 | B-12 | B-13 | — |
| monomer | monomer | Number of polymerizable functional | 1 | 1 | 2 | — | — | 1 | 1 | 1 | — |
| (B) | | group N₁ | | | | | | | | | |
| | | Molecular weight M₁ [g/mol] | 170 | 254 | 513 | — | — | 100 | 170 | 254 | — |
| | | Content W₁ [mass %] | 77 | 87 | 87 | — | — | 87 | 87 | 97 | — |
| | Cyclic | Type | — | — | — | B-22 | B-23 | — | — | — | B-21 |
| | ether | Number of polymerizable functional | — | — | — | 1 | 2 | — | — | — | 1 |
| | monomer | group N₂ | | | | | | | | | |
| | | Molecular weight M₂ [g/mol] | — | — | — | 130 | 340 | — | — | — | 72 |
| | | Content W₂ [mass %] | — | — | — | 87 | 87 | — | — | — | 87 |

| Photopolymerization | Type | IC907 | IC907 | IC907 | TTHFP | TTHFP | IC907 | IC907 | IC907 | TTHFP |
|---|---|---|---|---|---|---|---|---|---|---|
| inhibitor | Content [mass %] | 3 | 3 | 3 | 3 | 3 | 3 | 3 | 3 | 3 |
| Polymerization shrinkage coefficient α | | 9.06 | 6.86 | 6.78 | 6.69 | 1.28 | 17.40 | 10.24 | 7.64 | 12.08 |
| Protein non- | Protein adsorption rate P [%] | 0.05 | 0.1 | 0.1 | 0.1 | 0.1 | 0.1 | 0.1 | 0.8 | 0.1 |
| adsorption | Evaluation | ∘ | ∘ | ∘ | ∘ | ∘ | ∘ | ∘ | x | ∘ |
| Form stability | Warpage [mm] | 0.91 | 0.69 | 0.68 | 0.67 | 0.13 | 1.75 | 1.03 | 0.77 | 1.22 |
| | Evaluation | ∘ | ∘ | ∘ | ∘ | ∘ | x | x | ∘ | x |
| Cytotoxicity | Cell proliferation rate [%] | 17 | 98 | 173 | | | | 98 | 173 | |
| | Evaluation | x | ∘ | ∘ | | | | ∘ | ∘ | |

TABLE 2

| | | | Ex. 10 | Ex. 11 | Ex. 12 | Ex. 13 | Ex. 14 | Ex. 15 | Ex. 16 | Ex. 17 |
|---|---|---|---|---|---|---|---|---|---|---|
| Fluorinated | | Type | A-2 | A-2 | A-2 | A-2 | A-2 | A-2 | A-2 | A-2 |
| polymer | | Fluorine atom content Q_{F} [mass %] | 30.6 | 30.6 | 30.6 | 30.6 | 30.6 | 30.6 | 30.6 | 30.6 |
| (A) | | Content [mass %] | 20 | 10 | 10 | 10 | 10 | 10 | 10 | 10 |
| Curable | Vinyl | Type | B-12 | B-13 | B-14 | — | — | B-11 | B-12 | — |
| monomer | monomer | Number of polymerizable functional | 1 | 1 | 2 | — | — | 1 | 1 | — |
| (B) | | group N₁ | | | | | | | | |
| | | Molecular weight M₁ [g/mol] | 170 | 254 | 513 | — | — | 100 | 170 | — |
| | | Content W₁ [mass %] | 77 | 87 | 87 | — | — | 87 | 87 | — |
| | Cyclic | Type | — | — | — | B-22 | B-23 | — | — | B-21 |
| | ether | Number of polymerizable functional | — | — | — | 1 | 2 | — | — | 1 |
| | monomer | group N₂ | | | | | | | | |
| | | Molecular weight M₂ [g/mol] | — | — | — | 130 | 340 | — | — | 72 |
| | | Content W₂ [mass %] | — | — | — | 87 | 87 | — | — | 87 |

| Photopolymerization | Type | IC907 | IC907 | IC907 | TTHFP | TTHFP | IC907 | IC907 | TTHFP |
|---|---|---|---|---|---|---|---|---|---|
| inhibitor | Content [mass %] | 3 | 3 | 3 | 3 | 3 | 3 | 3 | 3 |
| Polymerization shrinkage coefficient α | | 9.06 | 6.86 | 6.78 | 6.69 | 1.28 | 17.40 | 10.24 | 12.08 |
| Protein non- | Protein adsorption rate P [%] | 0.03 | 0.05 | 0.05 | 0.05 | 0.05 | 0.05 | 0.05 | 0.05 |
| adsorption | Evaluation | ∘ | ∘ | ∘ | ∘ | ∘ | ∘ | ∘ | ∘ |
| Form stability | Warpage [mm] | 0.91 | 0.69 | 0.68 | 0.67 | 0.13 | 1.75 | 1.03 | 1.22 |
| | Evaluation | ∘ | ∘ | ∘ | ∘ | ∘ | x | x | x |
| Cytotoxicity | Cell proliferation rate [%] | 17 | 98 | 173 | | | | 98 | |
| | Evaluation | x | ∘ | ∘ | | | | ∘ | |

In Tables 1 and 2, blanks represent measurement was not conducted, and “-” represents no addition.

As shown in Tables 1 and 2, in each of Ex. 1 to 5 and 10 to 14 in which the protein adhesion inhibitor contained the fluorinated polymer (A) and the curable monomer (B) and had a coefficient α of at most 10, excellent protein non-adsorption was achieved, and the sheet as the cured product had a small warpage and excellent form stability. Whereas in Ex. 6, 7, 9 and 15 to 17 in which the coefficient α exceeded 10, the sheet as the cured product had a large warpage and was inferior in the form stability. Further, in Ex. 8 in which no fluorinated polymer (A) was contained, no sufficient protein non-adsorption was achieved.

This application is a continuation of PCT Application No. PCT/JP2017/019594, filed on May 25, 2017, which is based upon and claims the benefit of priority from Japanese Patent Application No. 2016-106136 filed on May 27, 2016. The contents of those applications are incorporated herein by reference in their entireties.

### REFERENCE SYMBOLS

1, 6: medical device, 2, 7: substrate, 3, 8: coating layer, 3a: convexo-concave pattern, 9: wetted part, 10: substrate sheet, 20: coating film, 22: cured film, 22a: convexo-concave pattern, 30: mold, 32: convexo-concave plane.

What is claimed is:

1. A protein adhesion inhibitor comprising a non-polymerizable fluorinated polymer having at least one group selected from the group consisting of a group represented by the following formula (1), a group represented by the following formula (2) and a group represented by the following formula (3) and having a fluorine atom content Q_{F} of from 5 to 60 mass %, and
at least one curable monomer selected from the group consisting of a vinyl monomer and a cyclic ether monomer,
wherein the coefficient α represented by the following formula (I) is at most 10: 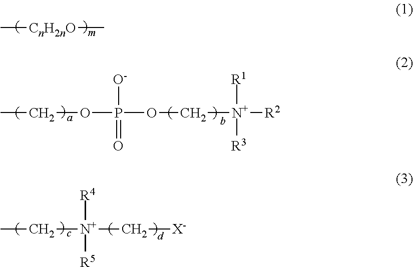
wherein n is an integer of from 1 to 10, m is an integer of from 1 to 100 in a case where the group represented by the formula (1) is contained in a side chain of the non-polymerizable fluorinated polymer or from 5 to 300 in a case where contained in the main chain, R¹ to R³ are each independently a C₁₋₅ alkyl group, “a” is an integer of from 1 to 5, b is an integer of from 1 to 5, R⁴ and R⁵ are each independently a C₁₋₅ alkyl group, X⁻ is a group represented by the following formula (3-1) or a group represented by the following formula (3-2), c is an integer of from 1 to 20, and d is an integer of from 1 to 5; 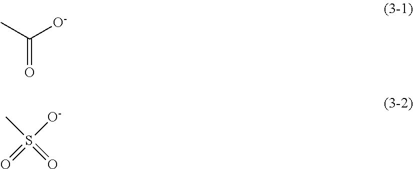
$$α = ( \frac{1000}{{M}_{1}} \times {N}_{1} \times \frac{{W}_{1}}{100} ) \times 2 + ( \frac{1000}{{M}_{2}} \times {N}_{2} \times \frac{{W}_{2}}{100} ) \qquad ( I )$$
wherein M₁ is the molecular weight of the vinyl monomer, N₁ is the number of polymerizable functional groups in the vinyl monomer, W₁ is the content (mass %) of the vinyl monomer to the total mass of the protein adhesion inhibitor, M₂ is the molecular weight of the cyclic ether monomer, N₂ is the number of polymerizable functional groups in the cyclic ether monomer, and W₂ is the content (mass %) of the cyclic ether monomer to the total mass of the protein adhesion inhibitor.

2. The protein adhesion inhibitor according to claim 1, wherein the content of the non-polymerizable fluorinated polymer is from 0.01 to 50.00 mass % to the total mass of the protein adhesion inhibitor.

3. The protein adhesion inhibitor according to claim 1, wherein the content of the curable monomer is from 50.00 to 99.99 mass % to the total mass of the protein adhesion inhibitor.

4. The protein adhesion inhibitor according to claim 1, which further contains a photopolymerization initiator which causes radical reaction or ionic reaction by light.

5. The protein adhesion inhibitor according to claim 4, wherein the content of the photopolymerization initiator is from 0.01 to 5.00 mass % to the total mass of the protein adhesion inhibitor.

6. The protein adhesion inhibitor according to claim 1, wherein the vinyl monomer is contained as the curable monomer, and the number of polymerizable functional groups in the vinyl monomer is from 1 to 20.

7. The protein adhesion inhibitor according to claim 1, wherein the vinyl monomer is contained as the curable monomer, and the molecular weight of the vinyl monomer is from 100 to 100,000.

8. The protein adhesion inhibitor according to claim 1, wherein the cyclic ether monomer is contained as the curable monomer, and the number of polymerizable functional groups in the cyclic ether monomer is from 1 to 20.

9. The protein adhesion inhibitor according to claim 1, wherein the cyclic ether monomer is contained as the curable monomer, and the molecular weight of the cyclic ether monomer is from 50 to 50,000.

10. A cured product of the protein adhesion inhibitor as defined in claim 1.

11. The cured product according to claim 10, which has a convexo-concave pattern formed on its surface.

12. A method for producing a cured product, which comprises irradiating a coating film formed from a coating solution containing the protein adhesion inhibitor as defined in claim 1 with light in such a state that a convexo-concave plane formed on the surface of a mold is pressed against the coating film, thereby to cure the coating film to obtain a film-form cured product.

13. An article having the cured product as defined in claim 10 on at least a part of its surface.

14. The article according to claim 13, which comprises a substrate and a coating layer formed of the cured product on the substrate.

15. The article according to claim 13, which is a medical device.
